(19) United States
(12) Patent Application Publication
(10) Pub. No.: US 2007/0078328 A1
(43) Pub. Date: Apr. 5, 2007

(54) Operation assisting system

(75) Inventors: Takashi Ozaki, Tokyo (JP); Akinobu Uchikubo, Iruma-shi (JP); Koichi Tashiro, Sagamihara-shi (JP); Takeaki Nakamura, Tokyo (JP)

Correspondence Address: Thomas Spinelli;Scully, Scott, Murphy & Presser, Suite 300, 400 Garden City Plaza, Garden City, NY, 11530, US

(73) Assignee: Olympus Corporation, Tokyo (JP)

(21) Appl. No.: 11/351,808

(22) Filed: Feb. 10, 2006

(30) Foreign Application Priority Data
Oct. 2, 2005 (JP) 2005-035168

(51) Int. Cl. A61B 5/05; A61B 1/04

(52) U.S. Cl. 600/407; 600/111

(57) ABSTRACT

A medical operation assisting system for displaying, based on medical image data, a virtual body-cavity image of a region on and surrounding a location to be operated within a body cavity of a subject, includes an image data generator for generating data of the virtual body-cavity image of the region on and surrounding the location to be operated, based on the medical image data, a storage device for storing optical characteristic data of an endoscope, and a controller for retrieving the optical characteristic data of the endoscope from the storage device, and causing the image data generator to generate the virtual body-cavity image data based on the optical characteristic data of the endoscope.

This application claims benefit of Japanese Patent Application No. 2005-035168 filed in Japan on Feb. 10, 2005, the contents of which are incorporated by this reference.

### BACKGROUND OF THE INVENTION

1. Field of the Invention

The present invention relates to medical operation assisting systems and, in particular, a medical operation assisting system using a three-dimensional virtual image as a reference image.

2. Description of the Related Art

With high-speed computers available, an endoscopic system becomes combined with a medical operation assisting system.

The medical operation assisting system reconstructs a volume rendering image (hereinafter simply referred to as rendering image) as a three-dimensional virtual image using medical image data in a three dimensional region, and displays, on a display screen of a monitor, a navigation image for guiding an endoscope or the like to a region of interest of a subject and a reference image for checking an area surrounding the region of interest.

Such a known medical operation system is applied to a broncho endoscope as disclosed in Japanese Unexamined Patent Application Publication No. 2000-135215.

The disclosed medical operation system generates a three-dimensional image of a tract of the subject based on the three-dimensional medical image data of the subject, determines a path to a target point along the tract on a three-dimensional medical image, generates a virtual rendering image of the tract along the path based on the medical image data, and displays the generated virtual rendering image on a monitor. The system thus navigates the broncho endoscope to a region of interest.

The medical operation system for use in the broncho endoscope displays the rendering image of the path specified beforehand, without a surgeon's operational command in the middle of an operation. The medical operation system is thus easy to be used, particularly, in the navigation of the broncho endoscope through a tract, such as a bronchial tract, along which a direction of view is limited.

In the known medical operation assisting system for use in surgical operations, a rendering image is displayed as a reference image in addition to an endoscopic image.

Surgeons typically perform surgical operations using a hand instrument such as an electrical knife while viewing an endoscopic image. The surgeon views a rendering image of a region surrounding a location of an organ to be operated to see blood vessels routed near the organ and the rear side of the organ.

In comparison with the navigation of the broncho endoscope, there is greater need for the medical operation assisting system to display a rendering image as a reference image the surgeon wants to see during an operation.

The known medical operation assisting system displays a rendering image when one of a nurse and an operator operates one of a mouse and a keyboard in response to an instruction from the surgeon.

### SUMMARY OF THE INVENTION

In one aspect of the present invention, a medical operation assisting system for displaying, based on medical image data, a virtual body-cavity image of a region on and surrounding a location to be operated within a body cavity of a subject, includes an image data generator for generating data of the virtual body-cavity image of the region on and surrounding the location to be operated, based on the medical image data, a storage device for storing optical characteristic data of an endoscope, and a controller for retrieving the optical characteristic data of the endoscope from the storage device, and instructing the image data generator to generate the virtual body-cavity image data based on the optical characteristic data of the endoscope.

In another aspect of the present invention, a medical operation assisting method of displaying, based on medical image data, a virtual body-cavity image of a region on and surrounding a location to be operated within a body cavity of a subject, includes steps of generating data of the virtual body-cavity image of the region on and surrounding the location to be operated, based on the medical image data, retrieving optical characteristic data of an endoscope from a storage device, and instructing an image data generator to generate the virtual body-cavity image data based on the retrieved optical characteristic data of the endoscope.

### BRIEF DESCRIPTION OF THE DRAWINGS

FIG. 1 illustrates a configuration of a medical operation assisting system in accordance with one embodiment of the present invention;

FIG. 2 is an external perspective view of an endoscope of FIG. 1;

FIG. 3 is a perspective view of the endoscope of FIG. 2 with a camera head attached to an eyepiece of the endoscope held by a surgeon;

FIG. 4 is an external perspective view of a trocar as an attaching object having a sensor mounted thereon;

FIG. 5 schematically illustrates a distal end portion of an insertion portion of a forward-viewing type endoscope;

FIG. 6 schematically illustrates a distal end portion of an insertion portion of an oblique-viewing type endoscope;

FIG. 7 is a block diagram of the medical operation assisting system of FIG. 1;

FIG. 8 is a flowchart illustrating operation of the medical operation assisting system of FIG. 1;

FIG. 9 illustrates a first display example of an endoscopic image;

FIG. 10 illustrates a display example of a virtual image corresponding to the endoscopic image of FIG. 9;

FIG. 11 illustrates a second display example an endoscopic image; and

FIG. 12 illustrates a display example of a virtual image corresponding to the endoscope image of FIG. 11.

### DETAILED DESCRIPTION OF THE PREFERRED EMBODIMENT(S)

An embodiment of the present invention is described below with reference to the drawings.

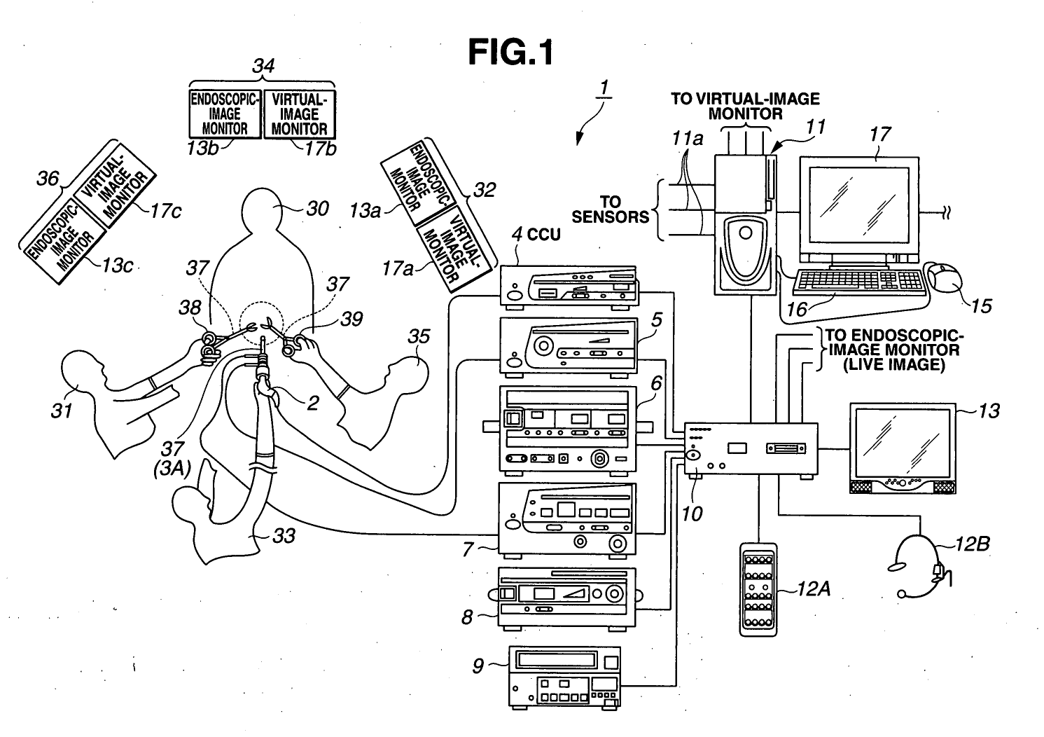

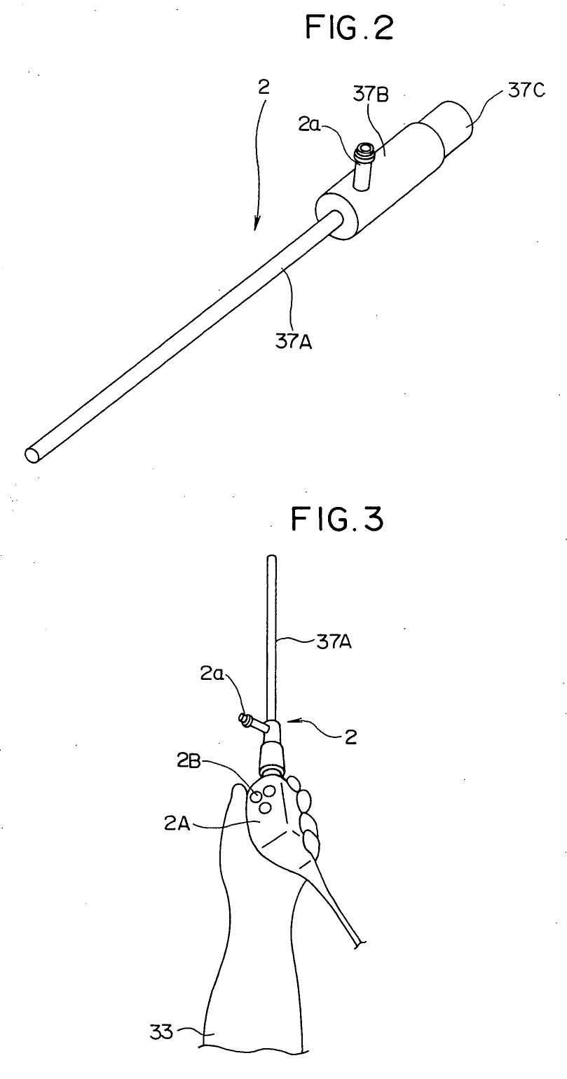

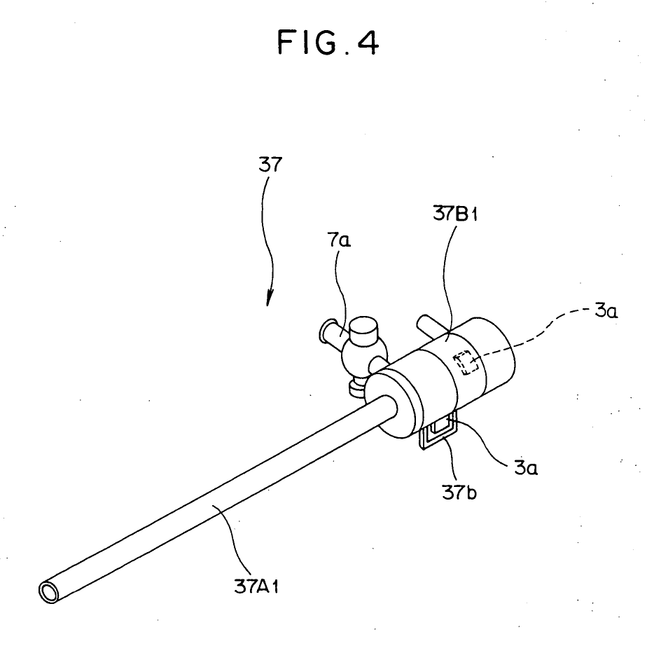

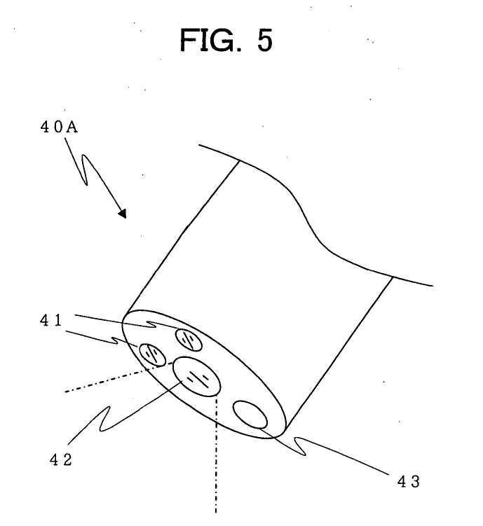

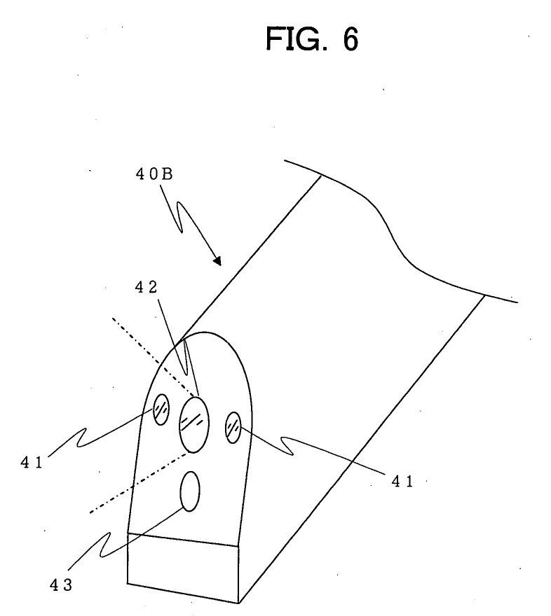

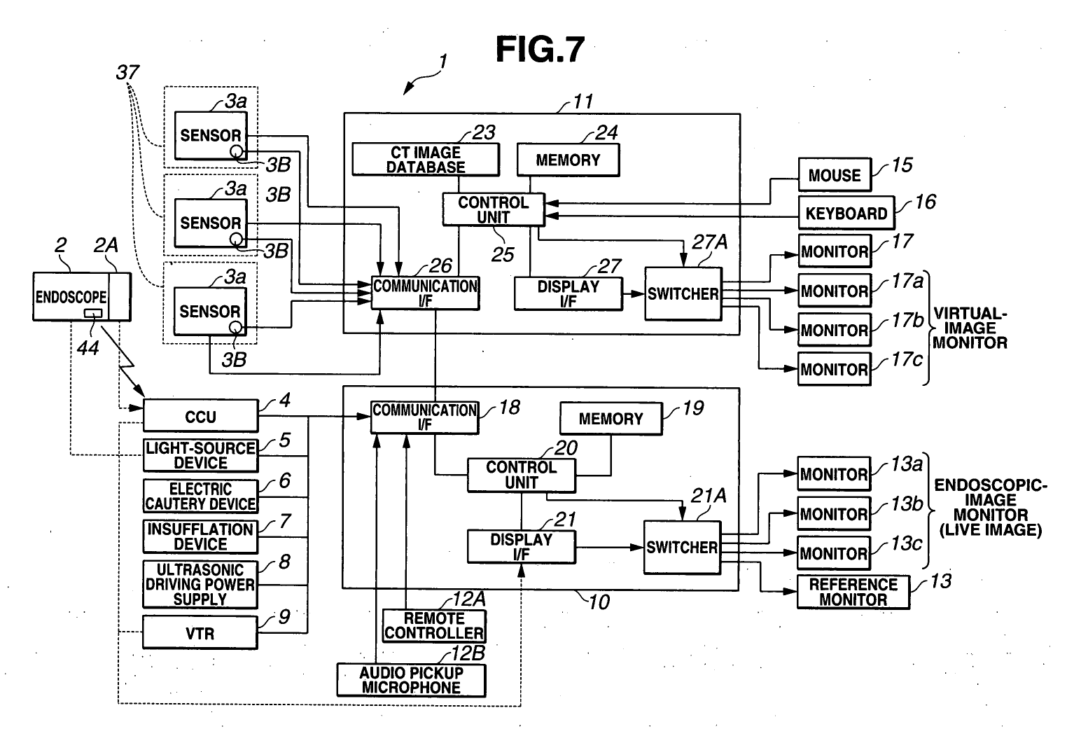

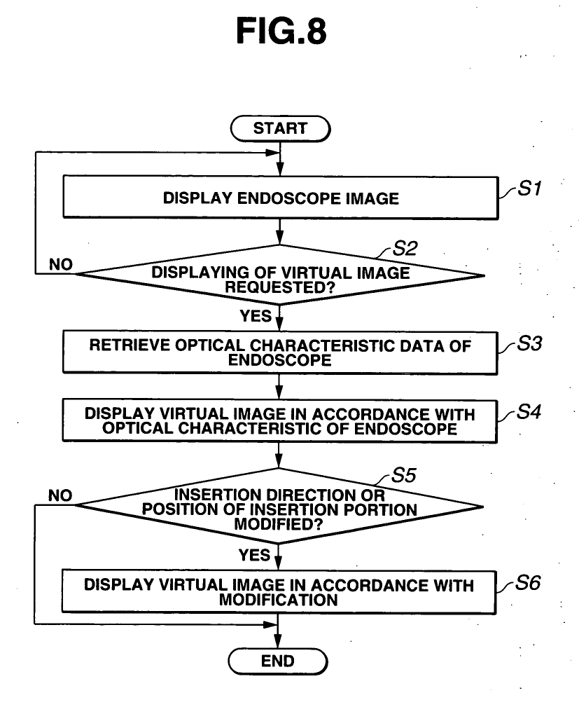

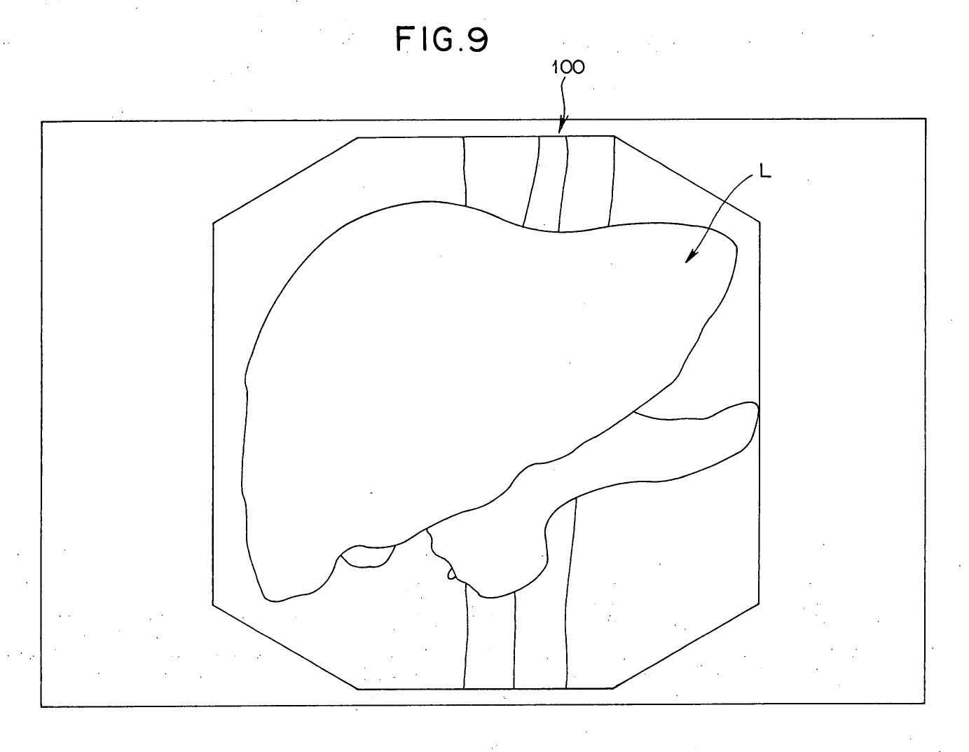

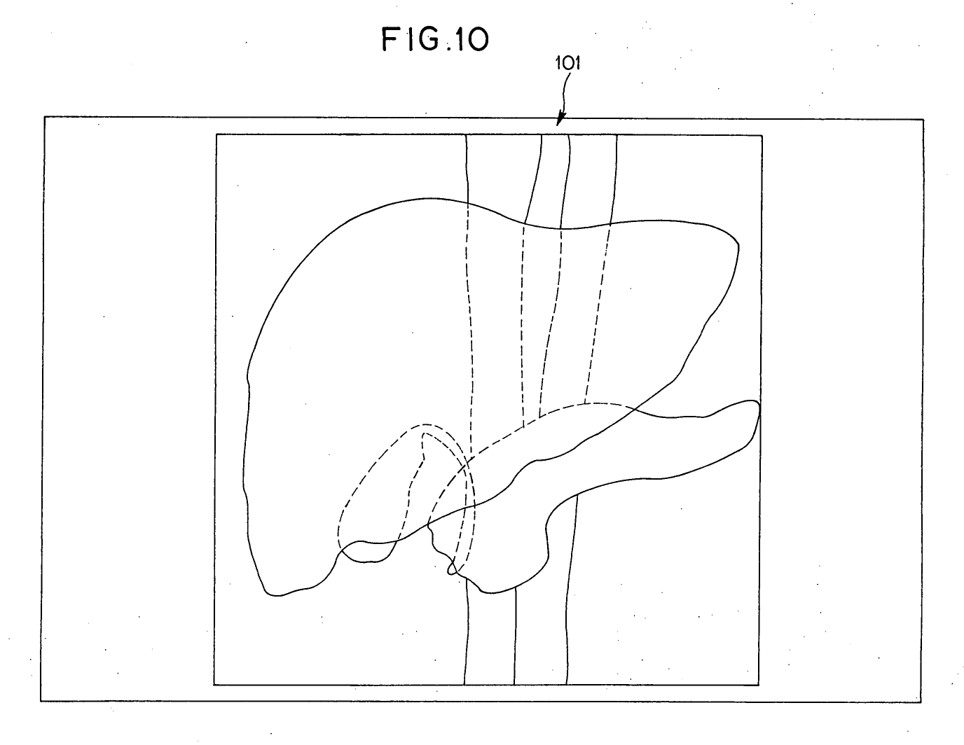

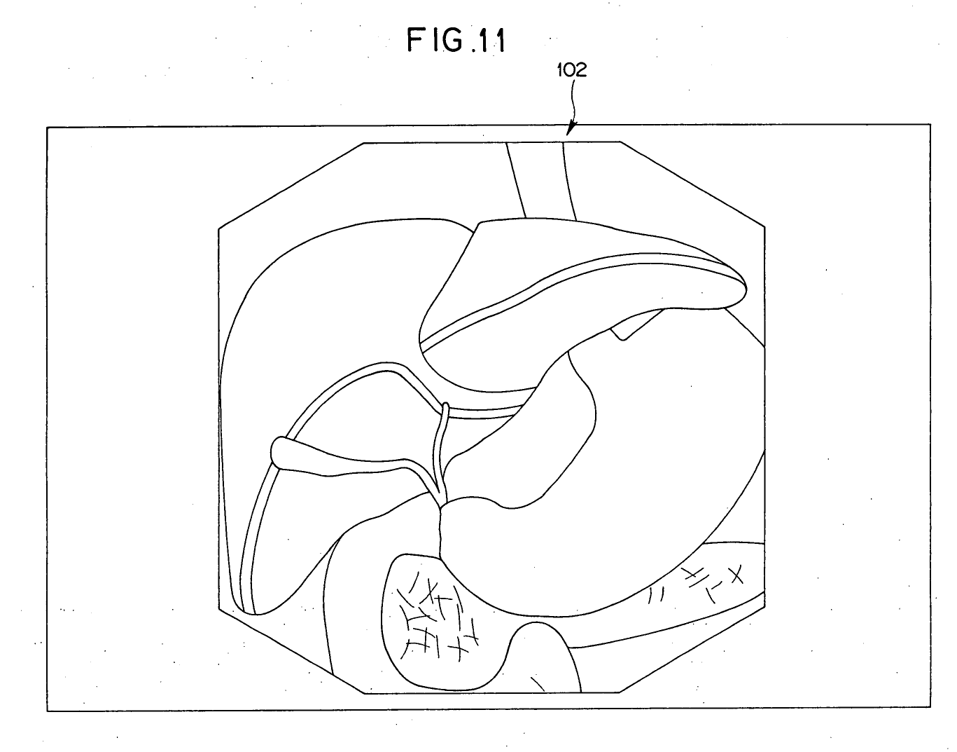

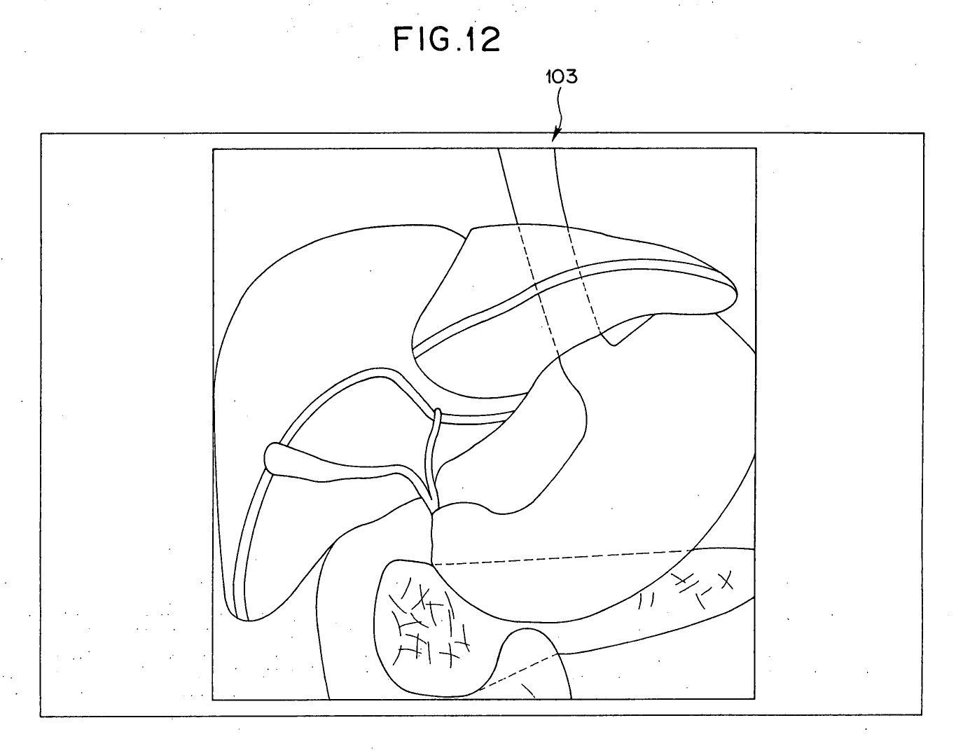

FIGS. 1 through 12 illustrate the embodiment of the present invention. FIG. 1 illustrates a configuration of a medical operation assisting system in accordance with one embodiment of the present invention, FIG. 2 is an external perspective view of an endoscope of FIG. 1, FIG. 3 is a perspective view of the endoscope of FIG. 2 with a camera head attached to an eyepiece of the endoscope held by a surgeon, FIG. 4 is an external perspective view of a trocar as an attaching object having a sensor mounted thereon, FIG. 5 schematically illustrates a distal end portion of an insertion portion of a forward-viewing type endoscope, FIG. 6 schematically illustrates a distal end portion of an insertion portion of an oblique-viewing type endoscope, FIG. 7 is a block diagram of the medical operation assisting system of FIG. 1, FIG. 8 is a flowchart illustrating operation of the medical operation assisting system of FIG. 1, FIG. 9 illustrates a first display example of an endoscopic image, FIG. 10 illustrates a display example of a virtual body-cavity image corresponding to the endoscopic image of FIG. 9, FIG. 11 illustrates a second display example an endoscopic image, and FIG. 12 illustrates a display example of a virtual body-cavity image corresponding to the endoscope image of FIG. 11.

As shown in FIG. 1, a medical operation assisting system 1 of one embodiment of the present invention is combined with an endoscope system. The medical operation assisting system 1 includes the endoscope 2 as observation means for observing the interior of the body cavity of a subject, at least two hand instruments, namely, a first hand instrument 38 and a second hand instrument 39, for handling the subject, an attaching object 3A (such as a trocar 37) for mounting sensors 3a respectively to the endoscope 2, the first and second hand instruments 38 and 39, a CCU 4 as an endoscopic image generator, a light-source device 5, an electric cautery device 6, an insufflation device 7, an ultrasonic driving power supply 8, a VTR (video tape recorder) 9, a system controller 10, a virtual image generator 11 serving as a virtual image generating device, a remote controller 12A, an audio pickup microphone 12B, a reference monitor 13 for displaying an endoscopic live image, a mouse 15, a keyboard 16, a monitor 17 for displaying a virtual image, and first-, second- and third-surgeon monitor devices 32, 34, and 36 arranged in an operating room.

The endoscope 2 is used as a laparoscope as shown in FIG. 2. This laparoscope includes an insertion portion 37A to be inserted into the body cavity of a subject, a grasping section 37B arranged at a proximal end of the insertion portion 37A, and an eyepiece section 37C arranged on the grasping section 37B.

An illumination optical system and an observation optical system are arranged within the insertion portion 37A. The illumination optical system and the observation optical system illuminate the interior of the body cavity of the subject, thereby resulting in an observation image of the intracavital region of the subject.

The grasping section 37B has a light-guide connector 2a. The light-guide connector 2a is connected to a connector attached to one end of a light-guide cable with the other end thereof connected to the light-source device 5. Light from the light-source device 5 via the illumination optical system in the endoscope 2 illuminates an observation region.

As shown in FIG. 3, the eyepiece section 37C can connect to a camera head 2A having a charge-coupled device (CCD) therewithin. The camera head 2A has a remote switch 2B for zooming in and out the observation image. A camera cable is extended and connected to the rear end of the camera head 2A. A connector (not shown) is attached to the other end of the camera cable for establishing an electrical connection to the CCU 4.

During a medical operation, the endoscope (laparoscope) 2 remains inserted into the trocar 37 as an attaching object, to which a sensor 3a to be described later is mounted. Furthermore, the trocar 37 receives, in addition to the endoscope 2, the first hand instrument 38 and the second hand instrument 39, to be respectively used by first and third surgeons 31 and 35.

In accordance with the present embodiment, the medical operation assisting system generates display data of a virtual body-cavity image with respect to the insertion direction of the endoscope 2 and the first and second hand instruments 38 and 39 to display the virtual body-cavity image. To this end, sensors 3a are mounted on the arms of the first through third surgeons 31, 33, and 35, and on the trocar 37 as the attaching object 3A through which the endoscope 2, and the first and second hand instruments 38 and 39 are inserted.

As shown in FIG. 4, the trocar 37 includes an insertion portion 37A1 to be inserted into the body cavity of the subject, a body 37B1 provided to the proximal end of the insertion portion 37A1, and an extension 37b extended from the outer circumference of the body 37B1.

An insufflation connector 7a is attached to the body 37B1. The insufflation connector 7a connects to a connector attached to one end of an insufflation tube with the other end thereof connected to the insufflation device 7. With this arrangement, the trocar 37 insufflates the peritoneal cavity by means of air supplied from the insufflation device 7, thereby assuring the field of view of the endoscope 2 and space within which the hand instruments are manipulated.

The sensor 3a having a switch 3B (FIG. 7) is loaded onto the extension 37b of the trocar 37. The sensor 3a may be secured on the outer circumference of the body 37B1 as outlined by broken lines in FIG. 4. Alternatively, the sensor 3a may be mounted on an extension portion that is detachably mounted on the outer circumference of the body 37B1.

The sensor 3a houses a sensor element such as a gyro sensor element, for example. The sensor 3a detects an insertion angle of the arm of a surgeon or the trocar 37 as the attaching object 3A with respect to the peritoneal cavity of the subject, and supplies information regarding the insertion angle and the like via a connection line (not shown in FIG. 4) to the virtual image generator 11 (FIG. 7).

Each sensor 3a is electrically connected to the virtual image generator 11 via respective connection line. Alternatively, each sensor 3a may be wirelessly linked to the virtual image generator 11 for data communication. The sensor 3a includes a press-button switch 3B that allows a surgeon to execute and switch display modes of a virtual image.

With the sensor 3a mounted on the trocar 37 in the present embodiment, the insertion direction of the endoscope 2 and the first and second hand instruments 38 and 39 approximately matches the insertion direction of the trocar 37. The sensor 3a thus acquires the information regarding the insertion angle and the like of the endoscope 2 and the first and second hand instruments 38 and 39.

During a medical operation, the endoscope 2 remains inserted through the trocar 37, and is held within the body cavity of the subject while the insertion portion 37A is inserted into the peritoneal cavity. The endoscope 2 picks up an endoscopic image of the peritoneal area of the subject via an objective optical system and an image pickup section such as a CCD in the camera head 2A. The image captured by the image pickup section is transferred to the CCU 4.

The endoscope 2 is different in optical characteristics such as in the direction of view and observation magnification depending on whether the endoscope 2 is a forward-viewing type endoscope 40A of FIG. 5 or an oblique-viewing type endoscope 40B of FIG. 6. Referring to FIGS. 5 and 6, there are shown an illumination optical system 41, an objective optical system 42, and a hand-instrument insertion channel opening 43.

The optical characteristics of the endoscope 2 include a direction of view, an angle of view, a depth of field, an observation distance, a range of view, observation magnification, etc. as listed in the following Table 1.

TABLE 1

| | Angle | Depth | | Range | |
|---|---|---|---|---|---|
| Direction | of | of | Observation | of | Observation |
| of view | view | field | distance | view | magnification |
| Forward- | 55° | 5 mm-∞ | 5 mm | 1.4 mm | 26-0.4 times |
| viewing | | | | dia. | |
| Oblique | 55° | 5 mm-∞ | 5 mm | 2.4 mm | 26-0.4 times |
| viewing | | | | dia. | |
| . . . | . . . | . . . | . . . | . . . | . . . |
| . . . | . . . | . . . | . . . | . . . | . . . |

In accordance with the present embodiment, image processing is performed to produce a virtual image based on the optical characteristics of the endoscope 2 as listed in Table 1.

As shown in FIG. 7, the endoscope 2 includes a Radio Frequency IDentification (RFID) tag 44 as a storage device storing optical characteristic data responsive to the type of the endoscope 2. The RFID tag 44 wirelessly transmits the optical characteristic data of an individual endoscope 2 to the CCU 4. The storage device storing the optical characteristic data of the endoscope 2 is not limited to the RFID tag 44. The storage device storing the optical characteristic data of the endoscope 2 may be a memory such as an integrated circuit (IC) memory. Alternatively, known endoscope identification means may be arranged and the storage device may be arranged in the CCU 4.

The CCU 4 processes the captured image signal, and supplies the system controller 10 in the operation room with image data (such as endoscopic live image data) derived from the captured image signal. Under the control of the system controller 10, the CCU 4 selectively outputs the image data of one of a still image and a moving image of the endoscopic live image to the VCR 9. The structure of the system controller 10 will be described later in more detail.

A receiver (not shown) of the CCU 4 receives the optical characteristic data of the endoscope 2 transmitted from the RFID tag 44 arranged in the endoscope 2. The CCU 4 then transmits the optical characteristic data to a control unit 20 in the system controller 10.

The light-source device 5 supplies illumination light to the endoscope 2 via a light guide. The electric knife probe (not shown) in the electric cautery device 6 cauterizes a lesion in the peritoneal region. An ultrasonic probe (not shown) in the ultrasonic driving power supply 8 cuts or coagulates the lesion. The insufflation device 7, including insufflation and aspiration means (not shown), sends a carbon dioxide gas to the body cavity region of the subject via the connected trocar 37.

The system controller 10 is electrically connected to and controls the light-source device 5, the electric cautery device 6, the insufflation device 7, and the ultrasonic driving power supply 8.

In addition to the above-described devices, the system controller 10 and the first-, second- and third-surgeon monitor devices 32, 34, and 36 are installed in the operating room.

The medical operation assisting system 1 of the present embodiment allows the three surgeons to perform the medical operation as shown in FIG. 1. More specifically, the first surgeon 31 operates the endoscope 2, the second surgeon 33 performs a clamping process, and the third surgeon 35 works as an assistant.

In the medical operation performed under observation of the endoscope 2, the first surgeon 31 performs a clamping process to a region of a subject 30 using the first hand instrument 38 such as a clamp, the second surgeon 33 operating the endoscope 2, and the third surgeon 35 assisting the first surgeon using the second hand instrument 39. The first, second and third surgeons 31, 33, and 35 perform their tasks at positions shown in FIG. 1.

In the present embodiment, the first-, second- and third-surgeon monitor devices 32, 34, and 36 are located at positions (in directions of view) easy for the first, second and third surgeons 31, 33, and 35 to see. More specifically, the first-surgeon monitor device 32, including an endoscopic-image monitor 13a and a virtual-image monitor 17a arranged side by side, is installed at a place where the first surgeon 31 can easily observe the first-surgeon monitor device 32. The second-surgeon monitor device 34, including an endoscopic-image monitor 13b and a virtual-image monitor 17b arranged side by side, is installed at a place where the second surgeon 33 can easily observe the second-surgeon monitor device 34. The third-surgeon monitor device 36, including an endoscopic-image monitor 13c and a virtual-image monitor 17c arranged side by side, is installed at a place where the third surgeon 35 can easily observe the third-surgeon monitor device 36.

The system controller 10 generally controls a variety of processes of the entire endoscope 2 (including display control and illumination control), and includes a communication interface (I/F) 18, a memory 19, a control unit 20 as control means, and a display interface (I/F) 21.

The communication I/F 18 electrically connects to the CCU 4, the light-source device 5, the electric cautery device 6, the insufflation device 7, the ultrasonic driving power supply 8, the VCR 9, and the virtual image generator 11 to be described later. Transmission and reception of drive control signals, and transmission and reception of endoscopic image data among these elements are controlled by the control unit 20. Furthermore, the communication I/F 18 electrically connects to the remote controller 12A for surgeon as remote control means and the audio pickup microphone 12B as an operation input unit. An operating instruction signal of the remote controller 12A and a voice instruction signal of the audio pickup microphone 12B are received via the communication I/F 18 and then supplied to the control unit 20.

The remote controller 12A includes a white balance button, an insufflation button, a pressure button, a video recording button, a freeze button, a release button, an image display button, a two-dimensional display control button, a three-dimensional display control button, an insertion point button, a point of interest button, a display magnification instruction button, a display color button, a tracking button, a decision execution button, and numerical keys, though these keys and buttons are not shown.

The white balance button is used to adjust white balance of images displayed on, for example, the endoscopic-image monitors 13a-13c, the virtual image display monitor 17, and the virtual-image monitors 17a-17c.

The insufflation button is used to drive the insufflation device 7. The pressure button is used to adjust intracavital pressure when the insufflation device 7 is operating. The video recording button is used to record an endoscopic live image. The freeze button is used to freeze the endoscopic image. The release button is used to release the freeze state of the image.

The image display button is used to display the endoscopic live image or the virtual image. The two-dimensional (2D) display control button is used to two-dimensionally display the virtual image. The 2D display control buttons include an axial button, a coronal button, and a sagittal button in accordance with a variety of 2D modes. The three-dimensional control button is used to display a three-dimensional (3D) virtual image.

The insertion point button is used to indicate insertion information of the endoscope 2 with respect to the peritoneal region, namely, the direction of view of the virtual image in a variety of 3D modes, such as the insertion point of the endoscope 2 in the peritoneal region represented in numeric values in X, Y, and Z directions. The point of interest button is used to indicate in numeric values the axial direction (angle) of the endoscope 2 when the endoscope 2 is inserted into the peritoneal region. The display magnification instruction button is used to instruct a modification in a display magnification in 3D display. The display magnification instruction buttons include a scale contraction button for contracting the display magnification, and a scale expansion button for expanding the display magnification.

The display color button is used to modify the color of display. The tracking button is used to perform a tracking process. The decision execution button is used to switch or determine input information set in an operation setting mode determined in response to the selection of each of the above-mentioned buttons. The numeric keys are used to input numerical values.

Using the remote controller 12A (or switch) having these buttons, the surgeons can operate the system to acquire quickly desired information.

The memory 19 stores the image data of the endoscopic still image, and data of device setting information. Storing and reading of these units of data are controlled by the control unit 20.

The display I/F 21 electrically connects to the CCU 4, the VCR 9, the reference monitor 13 and the endoscopic-image monitors 13a-13c. The display I/F 21 transmits and receives the endoscope live image data from the CCU 4 or the endoscopic image data played back by the VCR 9, and then outputs the received endoscopic live image data to the reference monitor 13 and the endoscopic-image monitors 13a-13c via the switcher 21A, for example.

The reference monitor 13 and the endoscopic-image monitors 13a-13c then display the endoscopic live image responsive to the endoscopic live image data.

Under the control of the control unit 20, the switcher 21A switches the endoscopic live image data as an output, thereby outputting the endoscopic live image data to any specified one of the reference monitor 13 and the endoscopic-image monitors 13a-13c.

Under the control of the control unit 20, the reference monitor 13 and the endoscopic-image monitors 13a-13c display, in addition to the endoscopic live image data, setting information regarding device setting statuses and parameters of the devices in the endoscopic system.

The control unit 20 performs a variety of control processes of the system controller 10, including transmission and reception control for transmitting and receiving a variety of signals through the communication I/F 18 and the display I/F 21, read and write control for reading image data from and writing image data to the memory 19, display control for displaying the images on the reference monitor 13 and the endoscopic-image monitors 13a-13c, and operation control responsive to operation signals from one of the remote controller 12A (or switch) and the switch 3B.

The system controller 10 is electrically connected to the virtual image generator 11. The virtual image generator 11 includes a computer tomography (CT) image database 23, a memory 24, a control unit 25 as an image data generator, a communication interface (I/F) 26, a display interface (I/F) 27, and a switcher 27A.

The CT image database 23 includes a CT image data acquisition unit (not shown) to acquire CT image data generated by a known CT apparatus (not shown) that captures an X-ray tomographic image of an intracavital operation region of a patient and an area surrounding the operation region. The CT image database 23 then stores the acquired CT image data. The CT image data acquisition unit can acquire the CT image data through a mobile storage device such as a magneto-optical (MO) drive or a digital versatile disk (DVD) drive. The read and write process of the CT image data are controlled by the control unit 25.

The memory 24 stores the CT image data, and data such as the virtual image generated by the control unit 25 from the CT image data. The store and read process of storing the data to and reading the data from the memory 24 are controlled by the control unit 25.

The communication I/F 26 is electrically connected to the communication I/F 18 in the system controller 10, the sensors 3a mounted on the attaching objects 3A of the first, second and third surgeons 31, 33, and 35, and the switch 3B. The communication I/F 26 transmits and receives control signals required for the virtual image generator 11 and the system controller 10 to operate in cooperation with each other. The transmission and reception of the control signals are controlled by the control unit 25 while the control signals are captured by the control unit 25.

The display I/F 27 outputs, to the virtual-image monitors 17, and 17a-17c via the switcher 27A, the virtual image of the operation region and the area surrounding the operation region, generated from the CT image data by the control of the control unit 25. The virtual-image monitors 17, and 17a-17c thus display the supplied virtual image. Under the control of the control unit 25, the switcher 27A switches the virtual images as an output, thereby to output the virtual image to a specified one of the virtual-image monitors 17, and 17a-17c. More specifically, the control unit 25 controls the selection as to which of the virtual-image monitors 17, and 17a-17c to display one or a plurality of generated virtual images. If there is no need for switching the virtual images, the switcher 27A may be eliminated. All the virtual-image monitors 17, and 17a-17c may display the same virtual image.

The control unit 25 is electrically connected to the mouse 15 and the keyboard 16, as operation devices. The mouse 15 and the keyboard 16 are used to input and/or set a variety of setting information required for the virtual-image monitors 17, and 17a-17c to display the virtual images.

The control unit 25 performs a variety of control process of the virtual image generator 11, including transmission and reception control of transmitting and receiving a variety of signals via one of the communication I/F 26 and the display I/F 27, read and write process of reading image data from and writing image to the memory 24, display control of the virtual-image monitors 17, and 17a-17c, switch control of the switcher 27A, and operation control to be performed in response to operation signals input from the mouse 15 and the keyboard 16.

The control unit 25 generates, as a rendering image, the virtual image responsive to the content of a medical operation. More specifically, the control unit 25 image processes the virtual image in accordance with the optical characteristic data of the endoscope 2. In accordance with the present embodiment, if the virtual image generator 11 is linked to a virtual image generator located at a remote location via communication means, a remote medical operation assisting system is formed.

The operation of the system of the present embodiment is described below.

When an observation image of the peritoneal region of the patient is captured by the camera head 2A in the medical operation assisting system 1, the endoscopic-image monitors 13a-13c display endoscopic images as shown in FIG. 8 (step S1).

A nurse, for example, initializes the medical operation assisting system 1 prior to the displaying of the virtual image. The nurse first enters information as to where the endoscope 2 is inserted in the abdomen of the patient (insertion position information in the abdomen represented in numerical values in X, Y, and Z directions) using one of the mouse 15 and the keyboard 16 while viewing the screen of the virtual-image monitor 17. The nurse also enters a numerical value of a point of interest in the axial direction of the endoscope 2 when the endoscope 2 is inserted into the abdomen. The nurse may further enter required information into the first hand instrument 38 and the second hand instrument 39 while viewing the screen thereof.

If the surgeon voices an instruction message “Display a virtual image” in the medical operation assisting system 1 in step S2 with the progress of the operation, the audio pickup microphone 12B detects the message in step S3. The control unit 20 in the system controller 10 recognizes the instruction message through a voice recognition process. More specifically, the voice recognized by the audio pickup microphone 12B is input to the control unit 20 as a voice signal, and the control unit 20 recognizes the voice through the voice recognition process thereof. As a result of voice recognition, the control unit 20 generates an instruction signal responsive to the instruction from the surgeon, and then commands the virtual image generator 11 to perform an image generation process of generating a virtual image.

The control unit 20 in the system controller 10 retrieves the optical characteristic data stored in the RFID tag 44 of the endoscope 2 via the CCU 4 (step S3). The control unit 20 commands the control unit 25 in the virtual image generator 11 to generate and display the virtual image responsive to the optical characteristic data.

In response to the input information, the control unit 25 in the virtual image generator 11 generates, based the CT image data, virtual images at the insertion point and the point of interest of the endoscope 2 and at the insertion point and the point of interest of the first and second hand instruments 38 and 39. In response to the control command from the control unit 20 in the system controller 10, the control unit 25 generates the virtual images in accordance with the optical characteristic data of the endoscope 2. More specifically, the virtual image generator 11 generates the virtual image based on position information of the distal end of the endoscope 2 in spatial coordinates determined based on the insertion points and the points of interest, the insertion axis direction of the endoscope 2, and the optical characteristic data.

As previously described, the optical characteristics of the endoscope 2 include the direction of view, the angle of view, the depth of field, the observation distance, the range of view, the observation magnification, etc. as listed in the Table. The control unit 25 generates the virtual image based on the optical characteristic data.

For example, if the observation magnification of the endoscope 2 is 5 times, the control unit 25 expands the virtual image by 5 times. If the direction of view of the endoscope 2 is 45°, the control unit 25 generates the virtual image in alignment with a direction of 45°.

The control unit 25 displays the generated virtual image on the virtual-image monitors 17, and 17a-17c. The monitor 17 mainly displays the virtual image corresponding to the endoscope 2. The monitor 17 may further display the virtual images from the first and second hand instruments 38 and 39.

FIG. 9 illustrates an endoscopic image 100 of a liver L and an area surrounding the liver L displayed on the virtual-image monitor 17, and FIG. 10 illustrates a virtual image 101 displayed on the virtual-image monitors 17a-17c.

The endoscopic-image monitors 13a, 13b, and 13c in the first-, second- and third-surgeon monitor devices 32, 34, and 36 for the first through third surgeons currently performing the operation show the endoscopic image of FIG. 9 under the display control of the control unit 20 in the system controller 10. The first through third surgeons 31 through 35 perform the operation while viewing the endoscopic image. In this case, the endoscope 2 and the first and second hand instruments 38 and 39 are used with the sensors 3a set on the trocar 37 as shown in FIG. 4.

During the operation, the control unit 25 in the virtual image generator 11 generates the virtual image based on the detection results from the sensors 3a of the endoscope 2 in a manner such that the virtual image matches the endoscopic image. The control unit 25 causes the monitor 17 and the virtual-image monitor 17b of the second-surgeon monitor device 34 to display the generated virtual image. Based on the detection results from the sensors 3a of the first and second hand instruments 38 and 39, the control unit 25 generates the virtual images corresponding to the two hand instruments. The control unit 25 then causes the virtual-image monitors 17a and 17b of the first-surgeon monitor device 32 and the third-surgeon monitor device 36 to display the generated virtual images.

During the operation, the second surgeon 33 now tilts the insertion section of the endoscope 2, thereby changing the angle of the axis or the position of the insertion section of the endoscope 2 with respect to the observation area of the intracavital region. In this case, as shown in FIG. 11, an endoscopic image 102 responsive to the angle of the axis of the endoscope 2 is displayed on the reference monitor 13 and the endoscopic-image monitors 13a-13c.

The sensor 3a detects the angle of the axis and the insertion position of the endoscope 2. The control unit 25 generates the virtual image based on the detection results of the sensor 3a. As shown in FIG. 12, the virtual image 103 is displayed on the monitor 17 and the virtual-image monitor 17b of the second-surgeon monitor device 34 (steps S5 and S6).

Likewise, the control unit 25 generates the virtual images based on the detection results of the sensors 3a for the first and second hand instruments 38 and 39. The virtual images derived from the first and second hand instruments 38 and 39 are respectively displayed on the virtual-image monitors 17a and 17c of the first-surgeon monitor device 32 and the third-surgeon monitor device 36.

When the axial angles and the insertion positions of the endoscope 2 and the first and second hand instruments 38 and 39 are changed, the virtual images corresponding to the endoscopic images are thus displayed respectively on the virtual-image monitors 17a-17c. The first through third surgeons 31, 33, and 35 thus acquire biological information of a subject in an observation area through an endoscopic observation image using the endoscope 2.

In accordance with the present embodiment, the rendering image matching the optical characteristics of the endoscope 2 is easily obtained.

The operation assisting system handles the 3D images in the above discussion. A filtering process of the present embodiment is applicable to the 2D images.

In accordance with the present embodiment, the medical operation assisting system is applied to an operation of the bile duct. The medical operation assisting system may be applied to other operations. For example, the medical operation assisting system of the present embodiment performs an image generation process in an operation of the duodenum.

The sensor 3a is attached to the trocar 37 in the above-described embodiment. In this case, the insertion point of the endoscope or the like is fixed. The virtual image is generated based on the spatial coordinates information of the endoscope or the like determined from the information regarding the fixed insertion point, and the information regarding the insertion axis direction (or the angle of insertion) of the trocar 37. Additionally, a sensor for detecting the length of insertion may be arranged. The position of the distal end of the endoscope or the like in the spatial coordinates is calculated from the insertion point and the length of insertion. The virtual image is generated using the spatial coordinates and the insertion axis direction.

The sensor 3a may be attached on the endoscope and the hand instrument rather than the trocar. The virtual image is generated using the position of the endoscope in spatial coordinates and the insertion axis direction of the endoscope.

An embodiment formed by combining part of the above-described embodiments also falls within the scope of the present invention.

The medical operation assisting system of the embodiment of the present invention easily generates the rendering image matching the optical characteristics of the endoscope.

The medical operation assisting system of the embodiment of the present invention is thus appropriate for use in observing the intracavital region of a patient by easily acquiring the rendering image matching the optical characteristics of the endoscope.

During operation, the known medical operation assisting system is accompanied by inconvenience and needs time when the surgeon attempts to explain a desired rendering image to nurses or operators.

In the known medical operation assisting system, the surgeon directly voices an instruction to a system controller using voice pickup means to control the entire system. To display a desired rendering image on a monitor, an operator must perform a complex operation. The desired rendering image cannot be displayed without an operator being skilled in rendering operation.

Depending on different type, the endoscopes are different in the optical characteristics such as the direction of view and the observation magnification, for example, from forward-viewing type to oblique-viewing type. In the known medical operation assisting system, the rendering image needs to be displayed taking into consideration the direction of view, the observation magnification, etc. of the endoscope. The operation of the system is thus complex.

In contrast, the medical operation assisting system of the embodiment of the present invention provides the rendering image matching the optical characteristics of the endoscope, and thus promotes the ease of use.

What is claimed is:

1. A medical operation assisting system for displaying, based on medical image data, a virtual body-cavity image of a region on and surrounding a location to be operated within a body cavity of a subject, comprising:
an image data generator for generating data of the virtual body-cavity image of the region on and surrounding the location to be operated, based on the medical image data;
a storage device for storing optical characteristic data of an endoscope; and
a controller for retrieving the optical characteristic data of the endoscope from the storage device, and causing the image data generator to generate the virtual body-cavity image data based on the optical characteristic data of the endoscope.

2. The medical operation assisting system according to claim 1, wherein the optical characteristic data comprises data regarding a direction of view and an angle of view of the endoscope.

3. The medical operation assisting system according to claim 1, wherein the image data generator generates the body-cavity image data based on information regarding spatial coordinates of the endoscope.

4. The medical operation assisting system according to claim 3, wherein the information regarding the spatial coordinates of the endoscope is acquired by a sensor mounted on an attaching object of the endoscope.

5. The medical operation assisting system according to claim 1, wherein the image data generator generates the body-cavity image data respectively for the endoscope and a hand instrument, based on the information regarding the spatial coordinates of the endoscope and the hand instrument.

6. The medical operation assisting system according to claim 5, wherein the information regarding the spatial coordinates of the endoscope and the hand instrument is acquired by sensors respectively mounted on attaching objects of the endoscope and the hand instrument.

7. The medical operation assisting system according to claim 6, wherein the controller controls a switcher, switching an output to two displays, to cause two pieces of the generated body cavity image data respectively from the endoscope and the hand instrument to be selectively displayed on the two displays.

8. The medical operation assisting system according to claim 1, wherein the image data generator generates the virtual body-cavity image data in response to an instruction signal generated as a result of recognition of a voice recognition operation.

9. A medical operation assisting method of displaying, based on medical image data, a virtual body-cavity image of a region on and surrounding a location to be operated within a body cavity of a subject, comprising steps of:
generating data of the virtual body-cavity image of the region on and surrounding the location to be operated, based on the medical image data;
retrieving optical characteristic data of an endoscope from a storage device; and
causing an image data generator to generate the virtual body-cavity image data based on the retrieved optical characteristic data of the endoscope.

10. The medical operation assisting method according to claim 9, wherein the optical characteristic data comprises data regarding a direction of view and an angle of view of the endoscope.

11. The medical operation assisting method according to claim 9, wherein the body-cavity image data is generated based on information regarding spatial coordinates of the endoscope.

12. The medical operation assisting method according to claim 11, wherein the information regarding the spatial coordinates is acquired by a sensor mounted on an attaching object of the endoscope.

13. The medical operation assisting method according to claim 9, wherein the body-cavity image data is generated based on the information regarding the spatial coordinates of the endoscope and a hand instrument.

14. The medical operation assisting method according to claim 13, wherein the information regarding the spatial coordinates of the endoscope and the hand instrument is acquired by sensors respectively mounted on attaching objects of the endoscope and the hand instrument.

15. The medical operation assisting method according to claim 14, wherein control operation is performed to cause two pieces of the generated body cavity image data from the endoscope and the hand instrument to be selectively displayed on two displays.

16. The medical operation assisting method according to claim 9, wherein the virtual body-cavity image data is generated in response to an instruction signal generated as a result of recognition of a voice recognition operation.
